(19) United States
(12) Patent Application Publication
(10) Pub. No.: US 2016/0175093 A1
(43) Pub. Date: Jun. 23, 2016

(54) OSSICULAR PROSTHESIS HAVING SPIKES

(71) Applicant: HEINZ KURZ GMBH MEDIZINTECHNIK, DUSSLINGEN (DE)

(72) Inventors: SEILESH BABU, NOVI, MI (US); UWE STEINHARDT, HIRRLINGEN (DE)

(21) Appl. No.: 14/972,264

(22) Filed: Dec. 17, 2015

(30) Foreign Application Priority Data
Dec. 19, 2014 (DE) 102014119224.9

(51) Int. Cl. A61F 2/18

(52) U.S. Cl. CPC A61F 2/18; A61F 2002/183; A61F 2220/0008; A61F 2220/0016; A61F 2220/0025

(57) ABSTRACT

An ossicular prosthesis comprising a sound-conducting prosthesis body formed with a first coupling element and a second coupling element. The second coupling element is designed as a clip or a slotted bell and has an access opening into a receiving space having an inner surface in the axial extension of the prosthesis body.

The inner surface is bounded by a peripheral outer edge having multiple interruptions. The second coupling element has, on an outer edge of the inner surface of the receiving space, at least two spikes distributed around the periphery of the outer edge, extending in a direction parallel to the axial extension of the elongate prosthesis body. In an implanted state, the spikes engage into the head of the stapes to effectuate a secure hold of the second coupling element.

### CROSS-REFERENCE TO A RELATED APPLICATION

The invention described and claimed hereinbelow is also described in German Patent Application DE 10 2014 119 224.9, filed on Dec. 19, 2014. The German Patent Application, the subject matter of which is incorporated herein by reference, provides the basis for a claim of priority of invention under 35 U.S.C. 119(a)-(d).

### BACKGROUND OF THE INVENTION

The present invention relates to an active or passive ossicular prosthesis, which is designed to replace or bridge at least one element of the human ossicular chain. The ossicular prosthesis comprises a sound-conducting, elongate prosthesis body, which has, on one end thereof, a first coupling element designed as a top plate for placement of the prosthesis against the tympanic membrane or as a clip for the mechanical connection to a component of the ossicular chain, in particular to the limb of incus or the manubrium of malleus, or is designed as a connecting piece for the sound-conducting connection to an actuator end piece of an active hearing implant. At its other end, the elongate prosthesis body comprises a second coupling element designed as a bell having several lateral slots or as a clip comprising a plurality of respectively alternating, lateral blades and slots, for a mechanical connection of the prosthesis to a stapes or to a head of stapes (“caput”). The second coupling element has an access opening into a receiving space having an inner surface in the axial extension of the elongate prosthesis body, which inner surface is bounded by a peripheral outer edge having multiple interruptions.

Ossicular prostheses are known from DE 10 2013 103484 B3, for example. Similar devices in the field of active hearing implants are described, for example, in U.S. Pat. No. 6,537,199 B1 or in DE 10 2010 046 457 B3. In passive ossicular prostheses, such arrangements having differently designed first and second coupling elements are used, for example, in U.S. Pat. No. A 5,514,177, in WO 98/16175 A1, in EP 1 181 907 B1, in DE 10 2008 015 117 B3 or, for example, in DE 10 2009 016 468 B3.

The human middle ear comprising the ossicles thereof has the function of transmitting the sound waves impacting the tympanic membrane via the external auditory meatus to the inner ear, which is filled with fluid. The three ossicles are the hammer (lat. malleus), which is attached to the tympanic membrane, the stirrup (lat.

stapes), which is connected via the footplate (lat. basis stapedis) thereof to the inner ear, and the anvil (lat. incus), which is located between the hammer and the stapes and is hingedly connected thereto.

Chronic middle ear inflammation is a disease of the human petrosal bone (=bone in which the entire ear is seated), in which degenerative processes can occur on the ossicular chain in a pathologically aggressive manner. As a result, the sound signal is not transmitted to the inner ear, or is transmitted incompletely, which results in conductive hearing loss.

Hearing implants are used to conduct the sound that impacts the auricle, or a corresponding sound signal, to the inner ear in cases in which the ossicles of the human middle ear are missing or damaged, in entirety or in part. A distinction is made between passive ossicular prostheses and active hearing implants. Passive ossicular prostheses physically replace parts of the ossicular chain, wherein sound is conducted “passively”, i.e. without the aid of powered auxiliary means. Active hearing implants receive powered signals corresponding to the sound signals from an amplifier by an actuator implanted in the middle ear. The amplifier is usually electronic and is associated with an externally or internally mounted hearing aid. Active hearing implants convert these powered signals at this point via mechanical motion back to acoustic oscillations and transmit the acoustic oscillations from a vibrating actuator end piece to the inner ear via a suitable connecting element.

Passive ossicular prostheses are used to improve sound transmission in patients having different pathologies. These passive ossicular prostheses are used to conduct sound from the tympanic membrane to the inner ear in cases in which the ossicles of the human middle ear are missing or damaged, either entirely or partially. The ossicular prosthesis has two ends. Depending on the specific circumstances, one end of the ossicular prosthesis is fastened to the tympanic membrane, e.g., using a top plate, and the other end of the ossicular prosthesis is fastened, e.g., to the stapes of the human ossicular chain, or it is inserted directly into the inner ear. In the known ossicular prostheses, sound conduction between the tympanic membrane and the inner ear is often limited because these known ossicular prostheses cannot fully replace the natural anatomical formations of the ossicular chain and the mechanisms of the middle ear, which have fine structures.

Three types of ossicular prostheses that are used particularly frequently are stapes prostheses, partial prostheses and total prostheses. Stapes prostheses are fixed to the incus and extend via a piston into the inner ear. Partial prostheses typically bear via a top plate against the tympanic membrane and establish a connection to the head of the stapes. Total prostheses connect the tympanic membrane to the base of stapes. The present invention relates exclusively to partial prostheses.

As shown clearly in the three, greatly enlarged photographs of more or less pathological human ear stapes bones in FIGS. 7a, 7b and 7c, the anatomical differences differ drastically in terms of shape and absolute size, and in the respective detailed situations specifically in the region of the head of the stapes. And it is precisely in the head of the stapes where the above-described partial prostheses are supposed to be coupled via the second coupling element thereof. A truly optimal coupling at this position would therefore require that the shaping of the particular coupling element on the implant that is used be adapted in an entirely individualized manner for each patient, which cannot be done, of course, at a reasonable expense.

### SUMMARY OF THE INVENTION

The present invention overcomes the shortcomings of known arts, such as those mentioned above.

The invention provides a markedly improved active or passive ossicular prosthesis, at a low cost and through a use of simple technical means in such a way that the above-described advantages of the known partial prostheses for handling in the region of the middle ear and the resultant improvement of sound transmission are retained, wherein, after implantation, however, the new markedly improved active or passive ossicular prosthesis design realizes a tilt-, slip- and wobble-resistant seat of the prosthesis on the upper region of the head of the stapes for very long periods of time. The new markedly improved active or passive ossicular prosthesis design also realizes additional degrees of freedom for the individualized adaptation to the anatomical circumstances of the specific patient in terms of shape, size and position of the patient's stapes bone.

The new markedly improved active or passive ossicular prosthesis design is particularly effective in that the second coupling element has, on the outer edge of the inner surface of the receiving space, at least two spikes. The at least two spikes are distributed around the periphery of the outer edge, extend in a direction parallel to the axial extension of the elongate prosthesis body and, in the implanted state of the ossicular prosthesis, engage into the stapes, in particular into the head of the stapes and, there, effectuate a secure hold of the second coupling element.

By the engagement of the spikes into the surface of the head of the stapes, a slip-resistant and positionally stable seat of the ossicular prosthesis on the stapes is made possible, also for very long time periods, in that sufficiently strong mechanical contact between the second coupling element and the head of the stapes is always realized. In addition, a tilt- and wobble-resistant lateral stabilization of the second coupling element in the end position thereof is achieved in this manner.

Due to the particularly durable mechanical seat of the prosthesis, which is easily ensured by the spikes provided according to the invention, the remaining geometrical shapes of the prosthesis are freely selected within relatively large limits and are exactly adapted and tuned to the specific conditions and anatomical circumstances of the specific patient.

When a slotted bell is used as the second coupling element, the slots in the bell have the advantage that the prosthesis can be slipped over the stapes even if the uppermost part of the stapes is missing. One variant of refinements of this embodiment is distinguished by the bell having a roundly arched bell hood. In many cases, however, refinements also are favorable in which the bell comprises a bell hood that is flattened and/or indented from above. This has the advantage that contact always occurs in the axial extension of the shank, thereby making it possible to clearly specify the length of the prosthesis.

An alternative class of embodiments is distinguished by the second coupling element designed as a clip comprising a plurality of respectively alternating, lateral blades and slots. This type of coupling element is often used in cases where particularly high stability is required intra-surgically, and therefore the surgeon utilizes a technically simple application.

In a simple embodiment of the ossicular prosthesis according to the invention, exactly two spikes are provided, which are disposed opposite one another on the outer edge, offset by 180° around the periphery. Even greater stability of the seat of the implanted ossicular prosthesis is expected in alternative embodiments, which are distinguished by exactly three spikes being provided. In this case, the three spikes are disposed opposite one another on the outer edge, offset by 120° with respect to one another around the periphery and, after implantation, effectuate, as it were, a “three-point seat” of the second coupling element on the head of the stapes.

The mechanical anchoring of the second coupling element of the ossicular prosthesis and, therefore, the positional stability of the postoperative seat thereof on the head of the stapes in the middle ear are very particularly high when exactly four spikes are provided. In this case, the four spikes are disposed on the outer edge so as to be offset by 90° with respect to one another around the periphery, and which are disposed opposite one another in pairs, so that the overall arrangement has high symmetry.

A class of embodiments of the ossicular prosthesis according to the invention includes that the spikes have a triangular shape, wherein a base of the triangle is fixedly connected to the outer edge of the inner surface of the receiving space and a tip of the triangle opposite the base protrudes from the inner surface. This simple shape of the spikes is also of great advantage in terms of a preferably uncomplicated producability of the prosthesis, since the actual dimensions of such details move within the range of a few micrometers.

In order to achieve increased flexibility and variability of the prosthesis, the elongate prosthesis body comprises at least one joint, in particular a ball joint, which has the advantage that the prosthesis thereby compensates for hydrostatic forces. Refinements are advantageous in terms of particularly high postsurgical mobility of the prosthesis in which a plurality of adjoining, further rotary elements are provided, preferably in the form of a ball joint chain, which makes it easy to vary the length of the prosthesis by pushing the ball chain through the joint receptacle and subsequently remove the overhanging, uppermost balls.

According to other refinements of these embodiments, the ball joint comprises a ball attached on the end of the prosthesis body thereof facing the second coupling element, a sleeve covering the ball on the side thereof remote from the second coupling element and a recess in the side facing the second coupling element, which functions as a socket for the ball. According to these embodiments, the ball joint is subjected to a soft support and therefore absorbs damping.

Preferred variants of these refinements are distinguished by the sleeve of the ball joint integrated into the prosthesis body being formed of a plastic sealing compound, preferably a silicone sealing compound.

In addition to the above-discussed problems of partial prostheses in the middle ear region, a notable detail problem is that, in the case of the common forms of coupling elements that are known and have been used for many years, for example in embodiments as a stapes bell, or when clips are used, a hollow space practically always forms as an extension of the shank-shaped prosthesis body between the inner side of the coupling element and the upper region of the head of the stapes, because the latter usually has a somewhat flattened shape, while the coupling elements typically used at this point are concavely arched.

A class of particularly advantageous embodiments of the ossicular prosthesis according to the invention is therefore distinguished by the second coupling element comprising a backing section on the inner surface of the receiving space, as an axial extension of the elongate prosthesis body. This backing section protrudes from the prosthesis body into the receiving space and, in the implanted state of the ossicular prosthesis, bears against the stapes, in particular against the head of the stapes. The backing section prevents or minimizes the formation of a hollow space between the stapes and the inner surface of the receiving space in the axial extension of the elongate prosthesis body. After implantation, the formation of a hollow space in the axial extension of the prosthesis body between the inner side of the coupling element and the upper region of the head of the stapes is reliably prevented, thereby improving sound conduction, because a hollow space, which obstructs sound, no longer forms therebetween. In addition, additional degrees of freedom are obtained for the individualized adaptation to the anatomical circumstances of the specific patient in terms of shape, size and position of the patient's stapes bone. In many refinements of this class of embodiments of the invention, the backing section is usually spherical or ellipsoid and is disposed symmetrically relative to the extended axis of the elongate prosthesis body, thereby ensuring that direct bearing contact with the stapes always occurs.

Other embodiments of the ossicular prosthesis according to the invention are also advantageous, however, for special situations and original shapes of the bone of the stapes in a patient:

According to an alternative refinement, for example, the backing section is conical and is disposed symmetrically relative to the extended axis of the elongate prosthesis body, wherein the cone tip protrudes from the elongate prosthesis body into the receiving space. This results in a highly reverberant connection, which is laterally affixed via point loading.

Refinements are also possible, however, in which the backing section is cylindrical and is disposed symmetrically relative to the extended axis of the elongate prosthesis body, wherein the cylinder protrudes from the elongate prosthesis body into the receiving space.

In other refinements, the backing section is plunger-shaped and is disposed symmetrically relative to the extended axis of the elongate prosthesis body, wherein a plunger shank carrying the plunger body protrudes from the elongate prosthesis body into the receiving space.

These refinements can be configured in different ways depending on the specific conditions of the patient, in particular, depending on the exact shape of the head of the stapes.

In a variant of these refinements, the plunger body has a concave contact surface directed into the receiving space, which engages directly even if the head of the stapes could have a convex shape.

Another variant is distinguished by the plunger body having a flat contact surface, which is directed into the receiving space.

In other cases, a variant also can be useful in which the plunger body has a convex contact surface directed into the receiving space, which engages directly even if the head of the stapes would have a concave shape.

The prosthesis will be designed according to the particular defect to be eliminated or at least ameliorated in terms of its effect on the patient via use of the ossicular prosthesis according to the invention.

In many embodiments of the invention, the first coupling element comprises a top plate designed to rest on the tympanic membrane. In other embodiments, the prosthesis can be attached on one side, for example, to the limb of incus or to the manubrium of malleus. In this context, an embodiment is advantageous in which the ossicular prosthesis is disposed at the end of the hammer (=umbo) or directly adjacent thereto, thereby resulting in the greatest leverage for the mechanical transmission of sound via motions that occur in the artificial or natural ossicular chain.

In addition to the postsurgical shifting of position, a further problem results once ossicular prostheses have been implanted. That is, the middle ear of the human body may be described as a “semi-open region”. Any implantation material that is inserted in the body within the scope of reconstruction of the middle ear and its structures thereby undergoes a particular stress which predominates in a contaminated and infected environment, and which typically attacks the material. Since an objective of implanting an ossicular prosthesis always is to enable the implant to remain in the patient's middle ear for as long as possible without complications occurring, a sustained attack on the material may result in damage being done to the prosthesis and/or in a local infection. Neither of these consequences is tolerable.

In a particularly preferred embodiment of the present invention, in order to permanently prevent damage from occurring to the implantation material or the surrounding tissue, the surface of the ossicular prosthesis is coated entirely or at least in sections with a biologically active coating, in particular, a growth-inhibiting and/or growth-promoting and/or antibacterial coating. A first coupling element, which is designed as a top plate, of the ossicular prosthesis according to the invention should always have a growth-promoting coating.

The ossicular prosthesis, according to the invention, or parts thereof may be made of titanium and/or gold and/or tantalum and/or steel, and/or an alloy of said metals. It is known that titanium, in particular, in addition to being stiff and having excellent sound-conducting properties, also exhibits excellent biocompatibility with the human middle ear.

Embodiments of the invention are very particularly advantageous in which the second coupling element is made, entirely or in part, of titanium or a material having a memory effect and/or super elastic properties, more particularly being made of Nitinol. Although the use of materials of this type is known per se in the field of ossicular prostheses, it proves particularly effective in conjunction with the present invention.

In terms of the postsurgical position adjustment described above, embodiments of the invention are advantageous in which the entire prosthesis or parts thereof, in particular the first coupling element as well, are made of a material having memory effect or super elastic properties, preferably being made of Nitinol, as is known per se, for example, from WO 02/069850 A1 or U.S. Pat. No. 6,554,861 B2.

As an alternative or in addition thereto, in further embodiments, parts of the ossicular prosthesis according to the present invention may be composed of a ceramic material. Embodiments of the present invention also are possible, however, in which the entire prosthesis or parts thereof are made of biocompatible plastics, particularly silicone, polytetrafluoroethylene (PTFE), or fibrous composite materials. By using these materials, is it possible to prevent post-operative rejection reactions from occurring in most cases.

### BRIEF DESCRIPTION OF THE DRAWINGS

Further features and advantages of the invention will become apparent from the description of embodiments that follows, with reference to the attached figures, wherein:

FIG. 1a shows a schematic spatial representation, diagonally from above, of a first embodiment of the ossicular prosthesis according to the invention, having a tympanic membrane top plate, as the first coupling element and a clip-shaped, second coupling element comprising a plurality of respectively alternating, lateral blades and slots;

FIG. 1b shows the embodiment of the ossicular prosthesis according to FIG. 1a, from below, as viewed into a receiving space of the second coupling element;

FIG. 1c shows the embodiment according to FIG. 1a, from the side;

FIG. 2a shows a schematic spatial representation, diagonally from above, of an embodiment of the ossicular prosthesis having a tympanic membrane top plate as a first coupling element and a bell having several slots, as the second coupling element, for placement against the head of the stapes;

FIG. 2b shows the embodiment of the ossicular prosthesis according to FIG. 2a, from below, as viewed into the receiving space of the second coupling element;

FIG. 2c shows the embodiment of the ossicular prosthesis according to FIG. 2a, from the side;

FIG. 3 shows a variable-length embodiment of the ossicular prosthesis according to the invention comprising a ball chain as the prosthesis body and four spikes on the bell-shaped, second coupling element and a backing section protruding into the receiving space of the second coupling element;

FIG. 4 shows an embodiment comprising a ball joint in the prosthesis body and a backing section as well as three spikes in the bell-shaped, second coupling element;

FIG. 5 shows a schematic cutaway representation of an embodiment of the ossicular prosthesis according to the invention in the region of a second coupling element, which is designed as a slotted bell, having a hemispherical backing section and a ball joint integrated into the end of the prosthesis body, wherein the backing section of the second coupling element is designed as a socket;

FIG. 6 shows an embodiment comprising a first coupling element, which is designed as a connecting piece for the sound-conducting connection to an actuator end piece of an active hearing implant, and a bell having several slots, as the second coupling element which has a backing section and two spikes;

FIG. 7a shows an image of a head of a human stapes;

FIG. 7b shows an image of a head of a human stapes, having the same magnification as the image of the head of the human stapes of FIG. 7a, where the stapes shown is different and pathologically altered as compared to that of the stapes of FIG. 7a; and

FIG. 7c shows an image of a head of a human stapes, having the same magnification as the image of the head of the human stapes of FIGS. 7a and 7b, where the stapes shown is different and pathologically altered as compared to that of stapes of FIGS. 7a and 7b.

### DETAILED DESCRIPTION OF THE PREFERRED EMBODIMENTS

The following is a detailed description of example embodiments of the invention depicted in the accompanying drawings. The example embodiments are presented in such detail as to clearly communicate the invention and are designed to make such embodiments obvious to a person of ordinary skill in the art. However, the amount of detail offered is not intended to limit the anticipated variations of embodiments; on the contrary, the intention is to cover all modifications, equivalents, and alternatives falling within the spirit and scope of the present invention, as defined by the appended claims.

The embodiments of the ossicular prosthesis 10; 20; 30; 40; 50; 60 according to the invention, which are represented schematically in the drawing figures and differ in terms of the detailed design thereof. The inventions each comprise, at one end, a first coupling element 11; 21; 31; 41; 61, which is used for the mechanical connection of the prosthesis to a component of the ossicular chain and is designed as the tympanic membrane top plate 11; 21; 31; 41 for placement against the tympanic membrane, or as a connecting piece 61 for the sound-conducting connection to an actuator end piece of an active hearing implant.

Seated at the other end of the ossicular prostheses 10; 20; 30; 40; 50; 60 is a second coupling element 12; 22; 32; 42; 52; 62, respectively, which comprises an access opening 14; 24 into a receiving space 15; 25 and is designed as a bell having several lateral slots 22; 32; 42; 52; 62 or as a clip 12 comprising a plurality of respectively alternating, lateral blades 12a and slots 12b, for a mechanical connection of the prosthesis to the stapes, in particular to the head of the stapes. Disposed therebetween is an elongate prosthesis body 13; 23; 33; 43; 53; 63 in the form of an elongate shank, which connects the two coupling elements 11; 21; 31; 41; 61 and 12; 22; 32; 42; 52; 62, respectively, to one another in a sound-conducting manner.

The receiving space 15; 25 has an inner surface in the axial extension of the elongate prosthesis body 13; 23; 33; 43; 53; 63, which inner surface is bounded by a peripheral outer edge 18; 28 having multiple interruptions.

According to the invention, the second coupling element 12; 22; 32; 42; 52; 62 is designed such that it comprises, on the outer edge 18; 28 of the inner surface of the receiving space 15; 25, at least two spikes 19; 29; 29′; 29″, which are distributed around the periphery of the outer edge 18; 28, extend in a direction parallel to the axial extension of the elongate prosthesis body 13; 23; 33; 43; 53; 63 and, in the implanted state of the ossicular prosthesis 10; 20; 30; 40; 50; 60, engage into the stapes, in particular into the head of the stapes and, there, effectuate a secure hold of the second coupling element 12; 22; 32; 42; 52; 62.

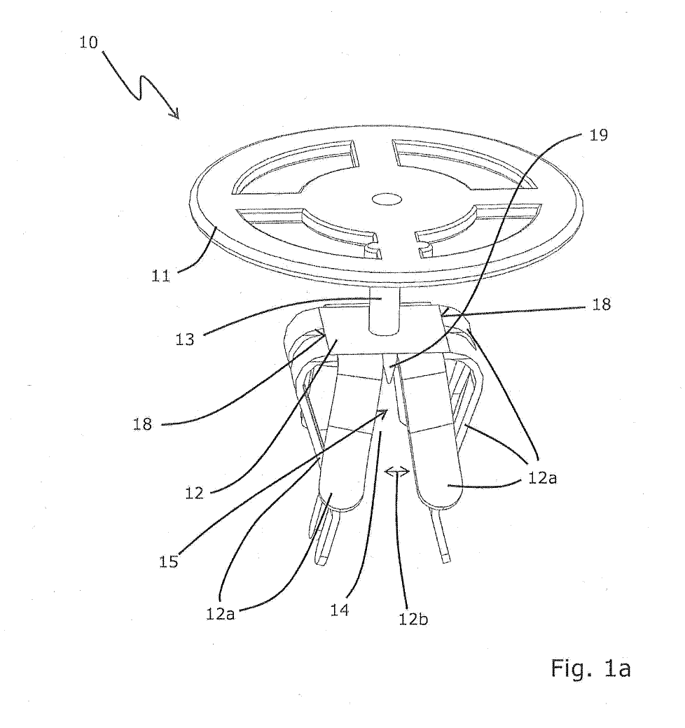

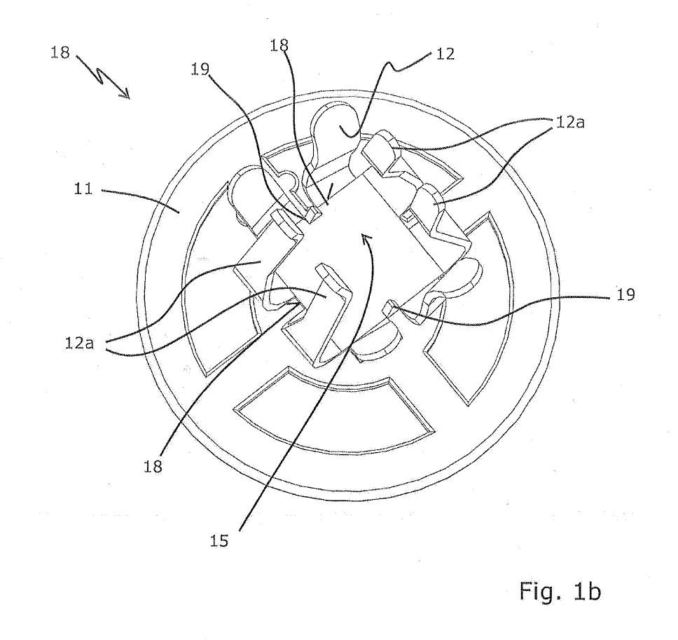

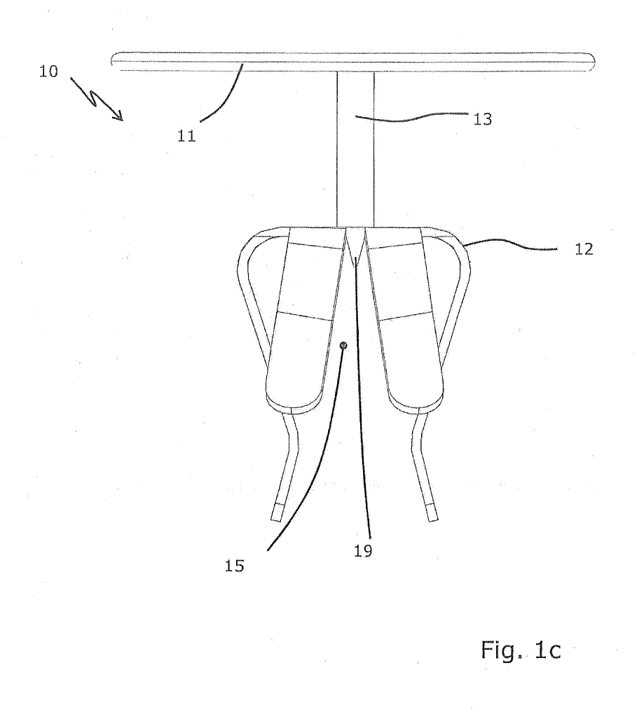

In all the embodiments represented in FIGS. 1a through 2c and 6, the second coupling element 12; 22; 62 comprises, on the outer edge 18; 28 of the inner surface of the receiving space 15; 25, exactly two spikes 19; 29 in each case, which spikes are disposed opposite one another, offset by 180° around the periphery.

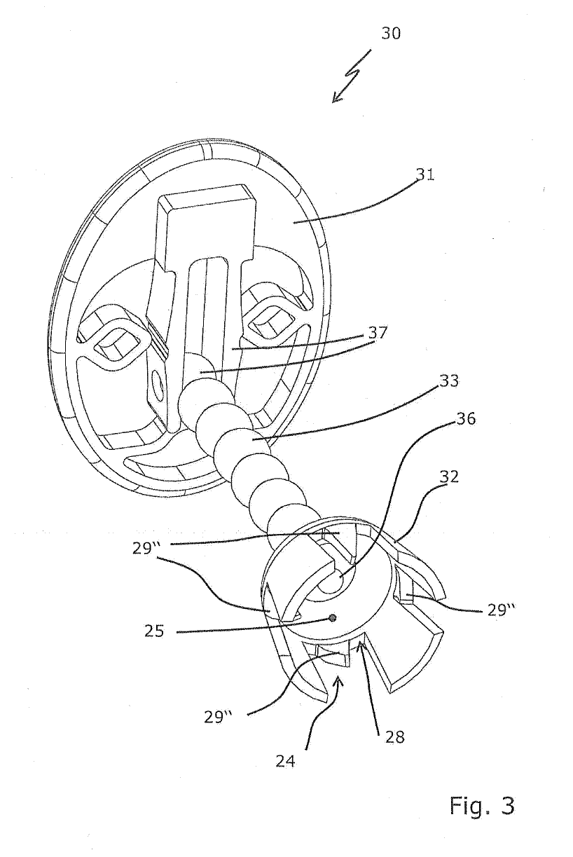

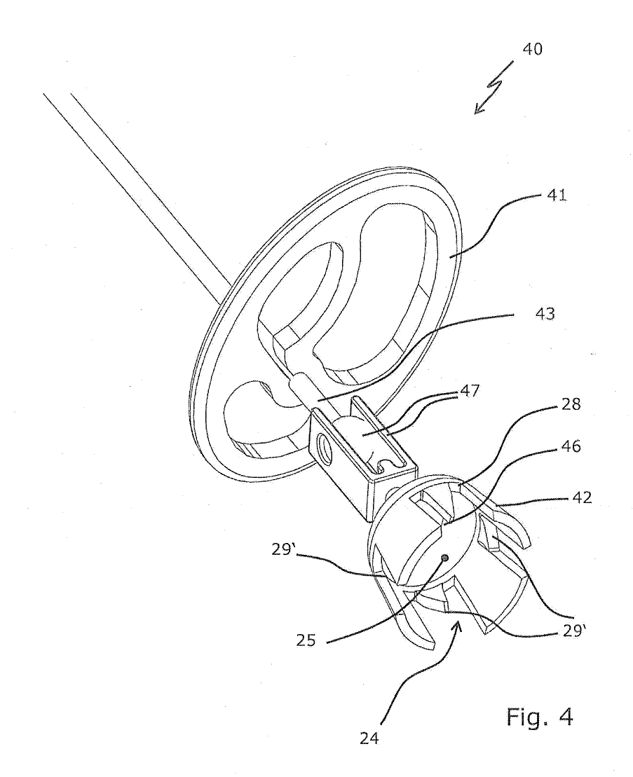

The embodiment according to FIG. 4 is distinguished by exactly three spikes 29′, which are disposed opposite one another on the outer edge 28, offset with respect to one another by 120° around the periphery. In the embodiment according to FIG. 3, exactly four spikes 29″ are provided, which are disposed on the outer edge 28 so as to be offset with respect to one another by 90° around the periphery and which are disposed opposite one another in pairs.

The spikes 19; 29; 29′; 29″ preferably have a triangular shape, wherein a base of the triangle is fixedly connected to the outer edge 18; 28 of the inner surface of the receiving space 15; 25 and a tip of the triangle opposite the base protrudes from the inner surface.

In the embodiment of the ossicular prosthesis 10 shown in FIGS. 1a through 1c, the first coupling element 11 is designed as a top plate for placement against the tympanic membrane. In this exemplary embodiment, the second coupling element 12 on the end of the elongate prosthesis body 13 opposite the top plate is designed as a clip comprising a plurality of respectively alternating, lateral blades 12a and slots 12b between the blades 12a.

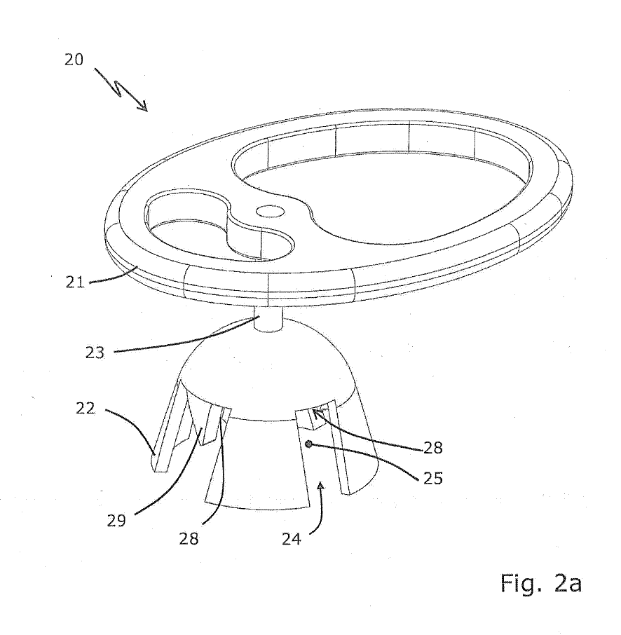

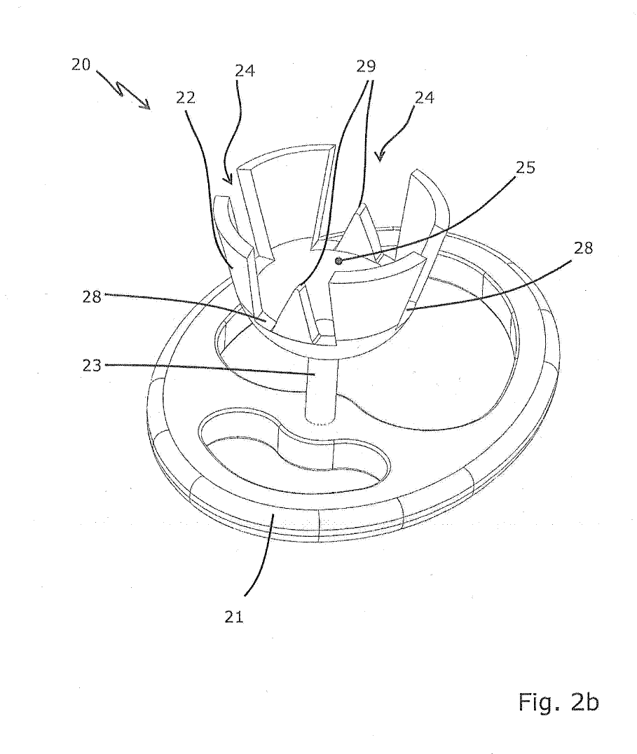

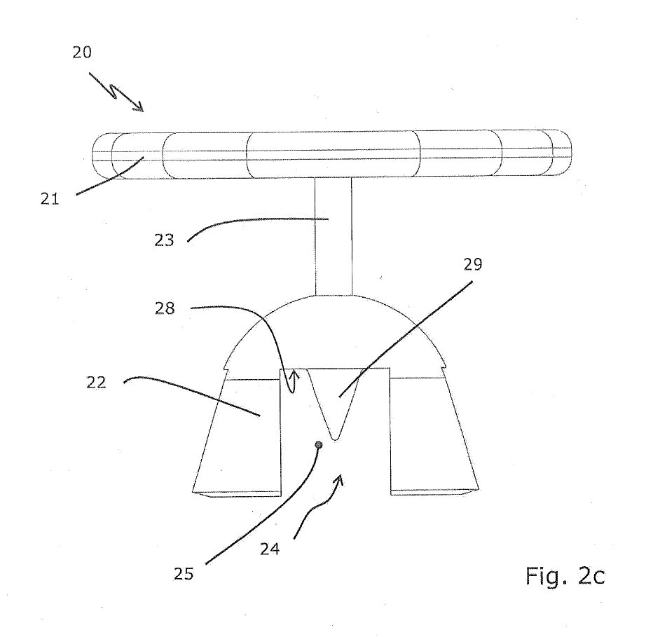

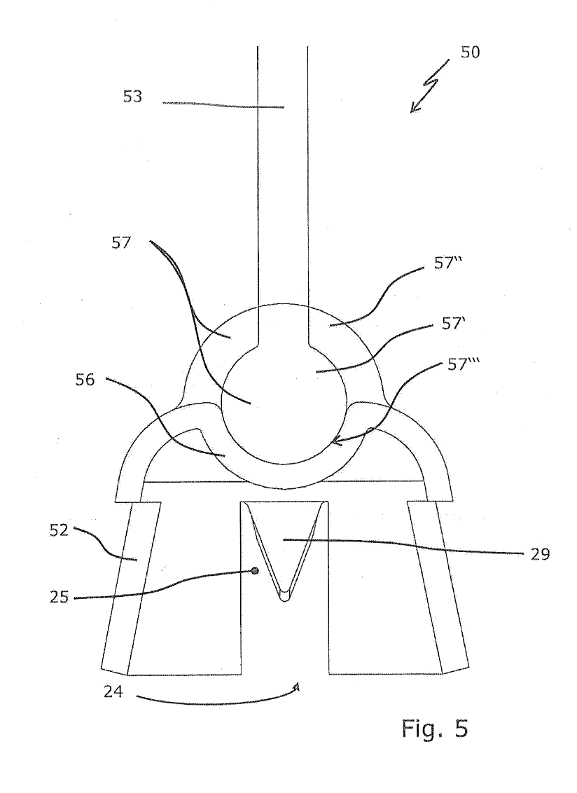

In the embodiments of FIGS. 2a through 2c, however, the first coupling element 21 is designed as a top plate for placement against the tympanic membrane. The second coupling element 22 is designed as a bell having several slots for placement against the head of the stapes and is fastened, via the bell hood, at the end of the elongate prosthesis body 23 opposite the first coupling element 21. The latter also applies for the embodiments represented in FIGS. 3 through 6. The bell-shaped coupling elements 22; 32; 42; 52; 62 in FIGS. 2a through 4 and 6 each comprise a roundly arched bell hood. The bell-shaped coupling element 52 in FIG. 5 comprises an indented bell hood. In the case of the embodiments of the invention not represented in the drawing, the bell-shaped coupling element can also comprise a flattened bell hood. The embodiments according to FIGS. 3 through 6 are characterized in that the second coupling element 32; 42; 52; 62 comprises a backing section 36; 46; 56; 66 on the inner surface of the particular receiving space 25, as an axial extension of the elongate prosthesis body 33; 43; 53; 63. This backing section projects from the elongate prosthesis body 33; 43; 53; 63 into the receiving space 25 and which, in the implanted state of the ossicular prosthesis 30; 40; 50; 60, bears against the stapes, in particular against the head of the stapes, and prevents or minimizes the formation of a hollow space between the stapes and the inner surface of the receiving space 25 in the axial extension of the elongate prosthesis body 33; 43; 53; 63.

In the embodiments of FIGS. 3 through 6 shown, the backing section 36; 46; 56; 66 is spherical or ellipsoid and is disposed symmetrically relative to the extended axis of the elongate prosthesis body 33; 43; 53; 63.

The embodiments represented in FIGS. 3 through 5 also are distinguished by the elongate prosthesis body 33; 43; 53 comprising at least one joint 37; 47; 57, in particular a ball joint.

Whereas the ball joint 37 of the ossicular prosthesis 30 according to FIG. 3 comprises an elongate prosthesis body 33 designed as a ball chain, the ball joint 47 in the ossicular prosthesis 40 in FIG. 4 is integrated into the shank-shaped, elongate prosthesis body 43. The ball chain of the ball joint 37 in FIG. 3 can be guided through a receiving section in the first coupling element 31, which is designed as a tympanic membrane top plate, and can be cut to the length required in the particular case. The remaining uppermost ball of the elongate prosthesis body 33 then forms a ball joint 37 together with the receiving section. In the embodiment according to FIG. 5, the ball joint 57 comprises a ball 57′, which is mounted on the end of the elongate prosthesis body 53 thereof facing the second coupling element 52, a sleeve 57″, which covers the ball 57′ on the side thereof remote from the second coupling element 52, and a recess 57″. The recess 57″ is indented in the outer surface of the bell hood of the second coupling element 52, wherein this recess functions as a socket for the ball 57′. The sleeve 57″ is formed from a plastic sealing compound, preferably from a silicone sealing compound. The inner side of the bell hood facing the receiving space 25 forms a hemispherical backing section 56 due to the above-described indentation.

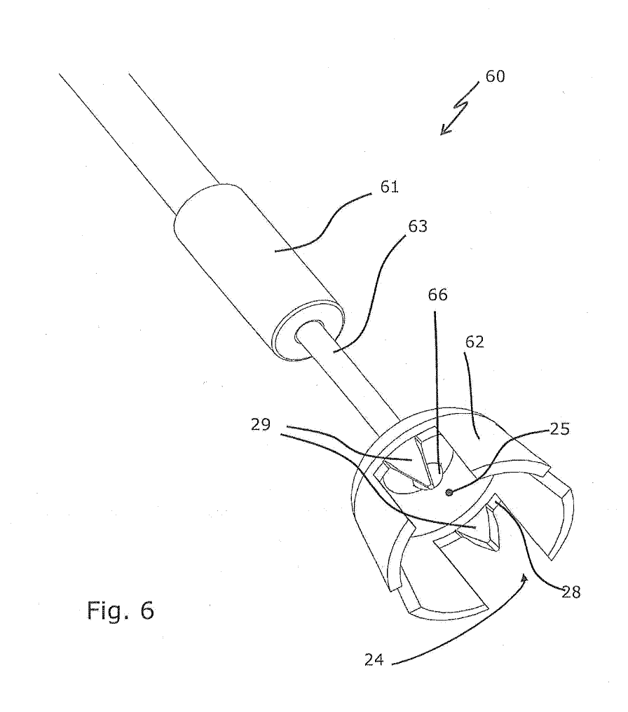

In the embodiment according to FIG. 6, the first coupling element 61 is designed as a connecting piece for the sound-conducting connection to an actuator end piece of an active hearing implant, which is not represented in greater detail in the drawing, whereas the second coupling element 62, however, has the shape of a bell having several slots.

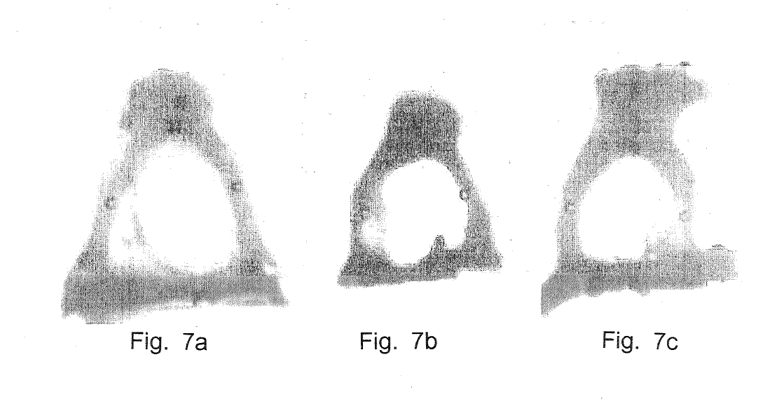

Finally, as mentioned above, FIGS. 7a, 7b and 7c shows three photographs of human stapes bones, each having the same magnification. Clearly shown are considerable differences in the geometric shape of the stapes, in particular, in the region of the head of the stapes. Also clearly shown are markedly different sizes of the particular stapes bone, and the details thereof, which are partially caused by pathological processes or are due simply to the natural, individual deviation between one patient and another. Due to the spikes on the second coupling element, which are provided according to the invention, and due to the various possible designs of the optional, additional backing section, optical sound conduction can be ensured specifically in the region of the head of the stapes, such as those shown.

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

What is claimed is:

1. An ossicular prosthesis designed to replace or bridge at least one element of the human ossicular chain, comprising:
a sound-conducting, elongate prosthesis body, having a first coupling element on one end and a second coupling element at another end;
wherein the first coupling element is designed as a top plate for placement of the prosthesis against a tympanic membrane, as a clip for mechanical connection to a component of the ossicular chain or as a connecting piece for the sound-conducting connection to an actuator end piece of an active hearing implant;
wherein the second coupling element is designed as a clip comprising a plurality of respectively alternating, lateral blades and slots or as a bell having several lateral slots, for a mechanical connection of the prosthesis body to a stapes or to a head of stapes; and
wherein the second coupling element has an access opening into a receiving space having an inner surface in an axial extension of the elongate prosthesis body, said access opening bounded by a peripheral outer edge having multiple interruptions; and
wherein the second coupling element has, on the outer edge of the inner surface of the receiving space, at least two spikes distributed around the periphery of the outer edge, extend in a direction parallel to the axial extension of the elongate prosthesis body and, in the implanted state of the ossicular prosthesis, engage into the stapes and, there, effectuate a secure hold of the second coupling element.

2. The ossicular prosthesis according to claim 1, wherein the at least two spikes are disposed opposite one another on the outer edge, offset by 180° around a periphery thereof.

3. The ossicular prosthesis according to claim 1, wherein three spikes are disposed opposite one another on the outer edge, offset with respect to one another by 120° around a periphery thereof.

4. The ossicular prosthesis according to claim 1, wherein four spikes are disposed opposite one another on the outer edge, offset with respect to one another by 90° around a periphery thereof, and wherein the four spikes are disposed opposite one another in pairs.

5. The ossicular prosthesis according to claim 1, wherein the at least two spikes have a triangular shape, wherein a base of the triangle-shaped spikes is fixedly connected to the outer edge of the inner surface of the receiving space and a tip of the triangle opposite the base protrudes from the inner surface.

6. The ossicular prosthesis according to claim 1, wherein the elongate prosthesis body comprises at least one joint.

7. The ossicular prosthesis according to claim 6, wherein the joint comprises a ball attached on the end of the elongate prosthesis body thereof facing the second coupling element, a sleeve covering the ball on the side thereof remote from the second coupling element, and a recess in the side facing the second coupling element, which functions as a socket for the ball.

8. The ossicular prosthesis according to claim 7, wherein the sleeve is formed from a plastic sealing compound.

9. The ossicular prosthesis according to claim 1, wherein the second coupling element further comprises a backing section on the inner surface of the receiving space, as an axial extension of the elongate prosthesis body, wherein the backing section protrudes from the prosthesis body into the receiving space and which, in the implanted state of the ossicular prosthesis, bears against the stapes and prevents or minimizes the formation of a hollow space between the stapes and the inner surface of the receiving space in the axial extension of the elongate prosthesis body.

10. The ossicular prosthesis according to claim 9, wherein the backing section is spherical or ellipsoid and is disposed symmetrically relative to the extended axis of the elongated prosthesis body.

11. The ossicular prosthesis according to claim 9, wherein the backing section is conical and is disposed symmetrically relative to the extended axis of the elongate prosthesis body, and wherein a cone tip protrudes from the elongate prosthesis body into the receiving space.

12. The ossicular prosthesis according to claim 9, wherein the backing section is cylindrical and is disposed symmetrically relative to the extended axis of the elongate prosthesis body, and wherein a cylinder protrudes from the elongate prosthesis body into the receiving space.

13. The ossicular prosthesis according to claim 9, wherein the backing section is plunger-shaped and is disposed symmetrically relative to the extended axis of the elongate prosthesis body, and wherein a plunger shank carrying the plunger body protrudes from the elongate prosthesis body into the receiving space.

14. The ossicular prosthesis according to claim 13, wherein the plunger body comprises a concave or flat contact surface directed into the receiving space.

15. The ossicular prosthesis according to claim 13, wherein the plunger body comprises a convex contact surface directed into the receiving space.
